(19) United States
(12) Patent Application Publication
(10) Pub. No.: US 2021/0121384 A1
(43) Pub. Date: Apr. 29, 2021

(54) METHOD OF PROTECTING HUMAN SKIN AGAINST DAMAGE UPON EXPOSURE WITH BLUE LIGHT

(71) Applicant: DSM IP ASSETS B.V., Heerlen (NL)

(72) Inventors: Christine MENDROK-EDINGER, Kaiseraugst (CH); Thomas RUDOLPH, Kaiseraugst (CH); Karolina STRAUSS, Kaiseraugst (CH)

(21) Appl. No.: 16/497,914

(22) PCT Filed: Mar. 26, 2018
(86) PCT No.: PCT/EP2018/057577

§ 371 Date: Sep. 26, 2019

(30) Foreign Application Priority Data
Mar. 31, 2017 (EP) 17164106.1

(51) Int. Cl. A61K 8/67; A61Q 17/04; A61K 8/06

(52) U.S. Cl. CPC A61K 8/675; A61K 2800/21; A61K 8/062; A61Q 17/04

(57) ABSTRACT

The present invention relates to a method for the protection of human skin against visible light damages, namely blue light, said method comprising the step of applying a cosmetic composition containing an effective amount of vitamin B3 or a derivative thereof and an effective amount of vitamin B6 or derivative thereof to the skin.

The present invention relates to a method for the protection of human skin against visible light damages.

The deleterious effects of skin exposure to ultraviolet (UV) radiation (both UVA and UVB) are well known. The UV radiation is categorized into three regions, UVC, UVB and UVA. Most of the UVC is filtered by ozone layer and high energy low wavelength UVB get absorbed in the upper layers of skin, i.e. epidermal region, while UVA penetrates little deeper into dermal regions of the skin. So far, most of the studies on skin exposure to light were concentrated on the role of UV irradiation due to its high energy, photo reactivity and its associated damage to the skin while the role of visible light has been less extensively investigated. In recent times, however, the adverse effects of visible light on skin has become more and more apparent.

Visible light is the region of light with 400-700 nm in the electromagnetic spectrum. Blue light is the portion of the electromagnetic spectrum in the visible region with wavelengths ranging from 400-500 nm. The wavelengths of blue light are close to UVA spectrum (315-400 nm) and the blue region of the visible spectrum is particularly important because it has a relatively high energy and at the same time longer wavelengths that can thus penetrate tissue deeper than UV light. It has recently been shown that blue light induces Reactive Oxygen Species (ROS) and Matrix-Degrading Enzymes in the skin which may lead inter alia to mitochondrial DNA damage and collagen degradation. Additionally, ROS generated by UV and blue light can cause further damage to proteins by oxidation of lysine, arginine, proline, and threonine residues. In addition, carbonyl groups may be introduced into proteins by reactions with aldehydes (4-hydroxy-2-nonenal, malondialdehyde) produced during lipid peroxidation or with reactive carbonyl derivatives generated as a consequence of the reaction of reducing sugars or their oxidation products with lysine residues of proteins. Both ROS and carbonyl groups on proteins (carbonylated proteins) can easily be measured in cells and tissue by a person skilled in the art and serve as markers for cell damage.

One defense system of the skin against reactive oxygen species (ROS) are radical scavenging substances like beta-carotene or lycopene which are also present in the skin. These antioxidants provide protection against ROS but they are themselves sensitive against electromagnetic irradiation and may thus serve as marker for skin damage.

Based on the more and more apparent adverse effects of electromagnetic radiation such as in particular visible light, there is an ongoing need for agents, which can protect the skin of the adverse effects of electromagnetic radiation such as in particular visible light.

Surprisingly, it has been found that the combination of niacinamide and pyridoxine hydrochloride synergistically protects the skin against the adverse effects of electromagnetic radiation such as in particular visible light.

Thus, in one aspect, the present invention relates to a method of protecting human skin against damage upon exposure to electromagnetic radiation, preferably visible light, more preferably blue light, said method comprising the step of applying a cosmetic composition containing an effective amount of Vitamin B₃ or a derivative thereof and Vitamin B₆ or a derivative thereof to the skin and optionally appreciating the effect.

In a still further embodiment, the present invention relates to the use of the combination of niacinamide and pyridoxine hydrochloride to protect human skin against the adverse effects of electromagnetic radiation, such as preferably of visible light, more preferably of blue light.

In an advantageous embodiment, the present invention relates to the use of a combination of niacinamide and pyridoxine hydrochloride to protect human skin against the formation of reactive oxygen species and/or light induced oxidative stress.

The term ‘electromagnetic radiation’ preferably refers to electromagnetic radiation between 290 nm and 3.0 μm, preferably between 290 nm to 800 nm, most preferably between 400 nm to 500 nm. Such radiation can be emitted from natural sources as well as from artificial devices such as electronic displays. More preferably, in all embodiments of the present invention, the term ‘electromagnetic radiation’ refers to visible light, most preferably to blue light.

The term ‘damage upon exposure to electromagnetic radiation as well as ‘adverse effects of electromagnetic radiation, such as in particular visible or even blue light’ as used herein encompasses in particular the generation of reactive species such as the formation of reactive oxygen species, reactive nitrogen species (RNS) and reactive carbonyl species (RCS) as well as the cellular damage thereof. Reactive species include radical and non-radical compounds such as nitroxyl-, carbonyl-, hydroxy- or oxyl-radicals, peroxylradicals, peroxides, superoxideradicalanion, hydrogenperoxide, singlet oxygen, lipoperoxides such as squaleneperoxides, ozone, nitrogene oxydes and NO-radical.

The term Vitamin B₃ and derivatives thereof as used herein preferable refers Niacinamide [CAS-Nr. 98-92-0], which is one of the water-soluble B-complex vitamins, niacin [CAS-Nr. 59-67-6], which is also known as nicotinic acid and nicotinamide riboside. Niacinamide is also referred to as nicotinamide or pyridine-3-carboxamide and is the amide of niacin (vitamin B₃) and is e.g. available as Niacinamide PC or Niacinamide from DSM Nutritional Products AG, (4303 Kaiseraugst, Switzerland). Particularly preferred in all embodiments of the present invention is the use of niacinamide.

The term Vitamin B₆ and derivatives thereof refers in particular to pyridoxine hydrochloride [58-56-0], pyridoxal [CAS-Nr. 66-72-8] and pyridoxamine [CAS-Nr. 85-87-0]. In all embodiments, particularly preferred is the use of pyridoxine hydrochloride also known as vitamin B₆ hydrochloride or vitamin B₆ which is e.g. available as Pyridoxine hydrochloride or Pyridoxine Hydrochloride 98 DC at DSM Nutritional Products AG, (4303 Kaiseraugst, Switzerland).

The term ‘an effective amount’ as used herein refers to an amount necessary to obtain the physiological effect. The physiological effect may be achieved by one application dose or by repeated applications. The dosage administered may, of course, vary depending upon known factors, such as the physiological characteristics of the cosmetic composition comprising the respective extract and its mode and route of administration; the age, the nature and extent of the symptoms; the kind of concurrent treatment; the frequency of treatment; and the effect desired and can be adjusted by a person skilled in the art.

Preferably, the amount of the Vitamin B₃ or a derivative thereof such as in particular niacinamide in the compositions according to the present invention is selected in the range of 0.5 to 10 wt. %, preferably in the range of 1 to 10 wt. %, most preferably in the range of 1 to 5 wt. %, based on the total weight of the composition.

Preferably, the amount of Vitamin B₆ or a derivative thereof such as in particular pyridoxine hydrochloride in the compositions according to the present invention is selected in the range of 0.02 to 6 wt. %, preferably in the range of 0.05 to 4 wt. %, most preferably in the range of 0.1 to 3 wt. %, based on the total weight of the composition.

In an advantageous embodiment, the amount of niacinamide is higher than the amount of pyridoxine hydrochloride. In a preferred embodiment, the ratio (w/w) of niacinamide to pyridoxine hydrochloride is selected in the range of 10 to 1 to 1.5 to 1, such as in the range of 5 to 1 to 2 to 1, such as 3 to 1.

In an advantageous embodiment, the compositions according to the present invention further comprise at least one UV-filter substance.

Suitable UV-filter substances according to the invention are UVA, UVB and/or broadspectrum UV-filter substances which are or can be used as cosmetically acceptable UVA, UVB or broadspectrum UV-filter substances. Such UV-filter substances are e.g. listed in the CTFA Cosmetic Ingredient Handbook or in the Regulation (EC) No 1223/2009 of the European Parliament and of the Council.

Preferred UV-B filter substances to be incorporated into the compositions according to the invention encompass polysilicone-15, phenylbenzimidazol sulfonic acid, octocrylene, ethylhexyl methoxycinnamate, ethylhexyl salicylate, and/or homosalate.

Preferred broadband UV-filter substances encompass bis-ethylhexyloxyphenol methoxyphenyl triazine, 2-hydroxy-4-methoxy-benzophenon, methylene bis-benzotriazolyl tetramethylbutylphenol and/or titanium dioxide.

Preferred UVA-filter substances encompass butyl methoxydibenzoylmethane, diethylamino hydroxybenzoyl hexyl benzoate, 2,4-bis-[5-1(dimethylpropyl)benzoxazol-2-yl-(4-phenyl)-imino]-6-(2-ethylhexyl)-imino-1,3,5-triazine, zinc oxide and/or disodium phenyl dibenzimidazole tetrasulfonate.

In a particular advantageous embodiment, the compositions according to the present invention comprise at least 2, more preferably at least 3, most preferably at least 4 different UV-filter substances. Even more advantageously, the compositions according to the invention, in addition, comprise at least one UV-B filter substance and at least one UVA-filter substance.

In another particular advantageous embodiment, the at least one UV-filter substance present in compositions according to the invention is selected from the group consisting of polysilicone-15, phenyl benzimidazol sulfonic acid, octocrylene, ethylhexyl methoxycinnamate, ethylhexyl salicylate, homosalate, bis-ethylhexyloxyphenol methoxyphenyl triazine, methylene bis-benzotriazolyl tetramethylbutylphenol, titanium dioxide, butyl methoxydibenzoylmethane, diethylamino hydroxybenzoyl hexyl benzoate, disodium phenyl dibenzimidazole tetrasulfonate as well as mixtures thereof.

In a specific embodiment, the at least one UV-filter substance is selected from the group consisting of bis-ethylhexyloxyphenol methoxyphenyl triazine, polysilicone-15, butyl methoxydibenzoylmethane, ethylhexyl salicylate, octocrylene, homosalate, methylene bis-benzotriazolyl tetramethylbutylphenol as well as mixtures thereof.

In all embodiments of the invention, the amount of the UV-filter substances (i.e. the sum of all UV-filter substances present in the composition according to the present invention) is preferably selected in the range of 1 to 40 wt.-%, more preferably in the range of 5 to 35 wt.-% and most preferably in the range of 10 to 30 wt.-% based on the total weight of the topical sunscreen emulsion.

In an even more specific embodiment, the compositions according to the invention comprise a mixture of bis-ethylhexyloxyphenol methoxyphenyl triazine, polysilicone-15, butyl methoxydibenzoylmethane, ethylhexyl salicylate, octocrylene, methylene bis-benzotriazolyl tetramethylbutylphenol as sole UV-filter substances. In this case the total amount of the UV-filter substances preferably sums up to 15 to 25 wt %.

The term ‘cosmetic composition’ as used herein refers to compositions, which are used to treat, care for or improve the appearance of the skin and/or the scalp. Particularly advantageous cosmetic compositions according to the present invention are skin care preparations.

The term ‘cosmetically acceptable carrier’ (also referred to herein as carrier) refers to all vehicles/carriers conventionally used in cosmetic compositions, i.e. which are suitable for topical application to the keratinous tissue, have good aesthetic properties, are compatible with the actives present in the composition, and will not cause any unreasonable safety or toxicity concerns. Such carriers are well-known to one of ordinary skill in the art.

The exact amount of carrier will depend upon the actual level of the active ingredients and of any other optional ingredients that one of ordinary skill in the art would classify as distinct from the carrier (e.g., other active ingredients).

In an advantageous embodiment, the cosmetic compositions according to the present invention comprise from about 50% to about 99%, preferably from about 60% to about 98%, more preferably from about 70% to about 98%, such as in particular from about 80% to about 95% of a carrier, based on the total weight of the cosmetic composition.

In an advantageous embodiment, the carrier consists furthermore of at least 40 wt.-%, more preferably of at least 50 wt.-%, most preferably of at least 55 wt.-% of water, such as in particular of about 55 to about 90 wt.-% of water.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions may also comprise further cosmetically active ingredients conventionally used in cosmetic compositions. Exemplary active ingredients encompass skin lightening agents; agents for the prevention or reduction of inflammation; firming, moisturizing, soothing, and/or energizing agents as well as agents to improve elasticity and skin barrier.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the active ingredients as well as the excipients, diluents, adjuvants, additives etc. can, based on the desired product form and application, easily be determined by the skilled person. The additional ingredients can either be added to the oily phase, the aqueous phase or separately as deemed appropriate.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The cosmetic compositions according to the present invention can be in a wide variety of forms. Non limiting examples include simple solutions (e.g. aqueous, organic solvent, or oil based), emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. of oil-in-water-in oil (O/W/O) or water-in-oil-in-water (W/O/W) type) or pickering emulsions), as well as solid forms (e.g. hydrogels, alcoholic gels, lipogels, sticks, flowable solids, or amorphous materials).

These product forms may be used for a number of applications, including, but not limited to, hand and body lotions, facial moisturizers, anti-ageing preparations, make-ups including foundations, and the like. Any additional components required to formulate such products vary with product type and can be routinely chosen by one skilled in the art.

If the composition is an emulsion, such as in particular an O/W—, W/O—, Si/W—, W/Si—, O/W/O—, W/O/W— or a pickering emulsion, then the amount of the oily phase present in such cosmetic emulsions is preferably at least 10 wt.-%, such as in the range of 10 to 60 wt.-%, preferably in the range of 15 to 50 wt.-%, most preferably in the range of 15 to 40 wt.-%, based on the total weight of the composition.

In one embodiment, the compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the composition according to the invention is an O/W emulsion, then it contains advantageously at least one O/W— or Si/W-emulsifier selected from the list of, glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g. as Amphisol® A from DSM Nutritional Products Ltd.), diethanolamine cetyl phosphate (e.g. as Amphisol® DEA from DSM Nutritional Products Ltd.), potassium cetyl phosphate (e.g. as Amphisol® K from DSM Nutritional Products Ltd.), sodium cetearylsulfate, sodium glyceryl oleate phosphate, hydrogenated vegetable glycerides phosphate and mixtures thereof. Further suitable emulsifiers are polyalkylene glycol ethers, sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier. For example, PVP eicosene copolymer, acrylates/C10-30 alkyl acrylate crosspolymer, and mixtures thereof.

The at least one O/W, respectively Si/W emulsifier is preferably used in an amount of 0.5 to 10 wt. %, in particular in the range of 0.5 to 6 wt.-%, such as more in particular in the range of 0.5 to 5 wt.-%, such as most in particular in the range of 1 to 4 wt.-%, based on the total weight of the composition.

Particular suitable 01W emulsifiers to be used in the compositions according to the invention encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate as well as polyalkylene glycol ethers such as in particular polyethylene stearyl ethers such as Steareth-2 and Steareth 21.

A particular suitable class of O/W emulsifier to be used in the compositions according to the invention are the cetyl phosphates such as in a particular potassium cetyl phosphate available at DSM Nutritional Products under the tradename Amphisol K.

In one particular embodiment, the invention relates to compositions with all the definitions and preferences given herein in the form of O/W emulsions comprising an oily phase dispersed in an aqueous phase in the presence of at least one O/W emulsifier wherein the at least one O/W emulsifier is potassium cetyl phosphate.

The cosmetic compositions according to the present invention advantageously comprise a preservative. Particular suitable preservatives in all embodiments of the present invention are phenoxyethanol and ethylhexylglycerin as well as mixtures thereof. When present, the preservative is preferably used in an amount of 0.1 to 2 wt.-%, more preferably in an amount of 0.5 to 1.5 wt.-%, based on the total weight of the composition.

The compositions according to the invention in general have a pH in the range of 3 to 10, preferably a pH in the range of 4 to 8 and most preferably a pH in the range of 5 to 8. The pH can easily be adjusted as desired with suitable acids, such as e.g. citric acid, or bases, such as sodium hydroxide (e.g. as aqueous solution), triethanolamine (TEA Care), Tromethamine (Trizma Base) and Aminomethyl Propanol (AMP-Ultra PC 2000), according to standard methods in the art.

The amount of the compositions to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

The following examples are provided to further illustrate the compositions and effects of the present invention. These examples are illustrative only and are not intended to limit the scope of the invention in any way.

### EXAMPLE

Following the paper of T. Rudolph et. al. (SOFW-Journal, 140, 3-2014), beta-carotene (DSM, β-Carotene Crystalline, Lot Nr. WC01503161) was dissolved in o-xylene to a concentration of 0.5% (w/w) and was homogenously applied over a PMMA plate (Schonberg, sandblasted, 2 μm roughness) with a syringe. After 10 min drying (RT, in the dark) the formulation as outlined in table 2 were applied (2 μLcm⁻²) and distributed with a finger. Per formulation 3 replicates were produced and irradiated. Parallel 3 replicates with placebo cream (no active) were prepared which were also irradiated. The irradiation took place in an Atlas SunTester XLS+ with a total irradiance of 500 Wm⁻² with an irradiation dose of 3MED. On top of the PMMA plates a UV-cut-off filter has been placed. This UV-cut-off filter is a Schott GG400-3.

After irradiation, the respective PMMA plates were extracted with 50 mL isopropanol in an ultrasonic bath for 1 min and the residual amount of beta-carotene was photometrically quantified at 452 nm.

Then the protection (%) was determined using the following formula:

% Protection=([(Absorbance sample)*100]/[Absorbance placebo]−100)

(The absorbance at 452 nm after irradiation of the placebo (maximum degradation) was set to 0%).

TABLE 1
Results

| Sample (active) | Protection* |
|---|---|
| 1 (none, Placebo) | 0% |
| 2 (3% Niacinamide) | +12% |
| 3 (1% Pyridoxin HCl) | +10% |
| 4 (3% Niacinamid + 1% Pyridoxin HCl) | +58% |

*Increase in % versus placebo (irradiated)

As can be retrieved from table 1, the addition of the combination of niacinamide and Pyridoxin hydrochloride to the placebo formulation led to a synergistic protection of the beta-carotene from blue light degradation.

TABLE 2

| | Sample | | | |
|---|---|---|---|---|
| INCI name | 1 | 2 | 3 | 4 |
| BIS-ETHYLHEXYLOXYPHENOL METHOXYPHENYL | 1.5 | 1.5 | 1.5 | 1.5 |
| TRIAZINE | | | | |
| POLYSILICONE-15 | 1 | 1 | 1 | 1 |
| BUTYL METHOXYDIBENZOYLMETHANE | 2 | 2 | 2 | 2 |
| ETHYLHEXYL SALICYLATE | 5 | 5 | 5 | 5 |
| OCTOCRYLENE | 1.5 | 1.5 | 1.5 | 1.5 |
| DIISOPROPYL SEBACATE | 3 | 3 | 3 | 3 |
| DIMETHICONE | 2 | 2 | 2 | 2 |
| DICAPRYLYL ETHER | 2 | 2 | 2 | 2 |
| DICAPRYLYL ETHER | 2 | 2 | 2 | 2 |
| TITANIUM DIOXIDE, SILICA, DIMETHICONE | 3 | 3 | 3 | 3 |
| CETYL PHOSPHATE | 1.5 | 1.5 | 1.5 | 1.5 |
| STEARYL ALCOHOL | 3.15 | 3.15 | 3.15 | 3.15 |
| HYDROXYETHYL ACRYLATE/SODIUM | 0.5 | 0.5 | 0.5 | 0.5 |
| ACRYLOYLDIMETHYL TAURATE COPOLYMER | | | | |
| POLYACRYLATE CROSSPOLYMER-6 | 0.5 | 0.5 | 0.5 | 0.5 |
| PHENOXYETHANOL, ETHYLHEXYLGLYCERIN | 1 | 1 | 1 | 1 |
| SILICA | 3 | 3 | 3 | 3 |
| AQUA | Ad 100 | | | |
| PROPANEDIOL | 5 | 5 | 5 | 5 |
| TROMETHAMINE, AQUA | 0.86 | 0.86 | 0.86 | 0.86 |
| METHYLENE BIS-BENZOTRIAZOLYL | 8 | 8 | 8 | 8 |
| TETRAMETHYLBUTYLPHENOL, AQUA | | | | |
| NIACINAMIDE | | 3 | | 3 |
| PYRIDOXIN HCl | | | 1 | 1 |

1. A method of protecting human skin against damage upon exposure to blue light, said method comprising the step of applying a cosmetic composition containing an effective amount of Vitamin B₃ or a derivative thereof and an effective amount of Vitamin B₆ or a derivative thereof to the skin.

2. The method according to claim 1, wherein the Vitamin B₃ or derivative thereof derivative is niacinamide and the Vitamin B₆ or derivative thereof if pyridoxine hydrochloride.

3. The method according to claim 1, wherein the effective amount of the Vitamin B₃ or derivative thereof is selected in the range of 0.5 to 10 wt. %, preferably in the range of 1 to 10 wt. %, most preferably in the range of 1 to 5 wt. %, based on the total weight of the composition.

4. The method according to claim 1, wherein the effective amount of the Vitamin B₆ or derivative thereof is selected in the range of 0.02 to 6 wt. %, preferably in the range of 0.05 to 4 wt. %, most preferably in the range of 0.1 to 3 wt. %, based on the total weight of the composition.

5. A method according to claim 1, wherein the amount of niacinamide is higher than the amount of pyridoxine hydrochloride.

6. A method according to claim 1, wherein the damage is the result of the generation of reactive oxygen species, reactive nitrogen species and/or reactive carbonyl species.

7. The method according to claim 1, wherein the composition further comprises at least one UV-filter substance.

8. The method according to claim 7, wherein the at least one UV-filter substance is selected from the group consisting of polysilicone-15, phenyl benzimidazol sulfonic acid, octocrylene, ethylhexyl methoxycinnamate, ethylhexyl salicylate, homosalate, bis-ethylhexyloxyphenol methoxyphenyl triazine, methylene bis-benzotriazolyl tetramethylbutylphenol, titanium dioxide, butyl methoxydibenzoylmethane, diethylamino hydroxybenzoyl hexyl benzoate, disodium phenyl dibenzimidazole tetrasulfonate as well as mixtures thereof.

9. The method according to claim 1, wherein the composition is a skin care composition.

10. The method according to claim 1, wherein the composition is in the form of O/W emulsions comprising an oily phase dispersed in an aqueous phase in the presence of an O/W emulsifier.

11. The method according to claim 10, wherein the O/W emulsifier is potassium cetyl phosphate.

12. A method of reducing the generation of reactive oxygen species and/or the formation of carbonylated proteins in humans when exposed to blue light, said method comprising the step of applying a cosmetic composition containing an effective amount of niacinamide and pyridoxine hydrochloride to the skin.

13. A method according to claim 1, wherein the effective amount of the niacinamide in the cosmetic composition is selected in the range of 0.5 to 10 wt. %, and the amount of pyridoxine hydrochloride is selected in the range of 0.02 to 6 wt. %, based on the total weight of the cosmetic composition.

14. A combination of niacinamide and pyridoxine hydrochloride for the use in the protection of human skin against the adverse effects of electromagnetic radiation.

15. The use according to claim 14, wherein the adverse effects are the formation of reactive oxygen species and/or light induced oxidative stress.
